(19) United States
(12) Patent Application Publication
(10) Pub. No.: US 2014/0213936 A1
(43) Pub. Date: Jul. 31, 2014

(54) DEVICES, SYSTEMS, AND METHODS FOR TISSUE MEASUREMENT

(71) Applicant: Monolysis Medical, LLC, Lexington, MA (US)

(72) Inventors: Demetri A. Monovoukas, Lexington, MA (US); Michael J. Monovoukas, Lexington, MA (US)

(21) Appl. No.: 14/090,635

(22) Filed: Nov. 26, 2013

Related U.S. Application Data
(60) Provisional application No. 61/757,797, filed on Jan. 29, 2013.

(51) Int. Cl. A61B 5/107

(52) U.S. Cl. CPC A61B 5/1077
USPC 600/587

(57) ABSTRACT

Devices, systems, and methods for tissue measurement are provided. In general, the devices, systems, and methods can allow a size of a target tissue, e.g., a wound, of a patient to be determined by tracing a perimeter of the target tissue using a handheld device. Examples of the size of the target tissue include a length of a perimeter of the target tissue, a surface area of the target tissue, a depth of the target tissue, and a volume of the target tissue.

### CROSS REFERENCE TO RELATED APPLICATIONS

This application claims priority to U.S. Prov. Pat. App. No. 61/757,797 entitled “Hand Held Device Used To Measure Dimensions Of A 2D Or 3D Shape” filed Jan. 29, 2013, which is hereby incorporated by reference in its entirety.

### FIELD

The present disclosure relates generally to devices, systems, and methods for tissue measurement.

### BACKGROUND

Wounds such as pressure ulcers, also known as bedsores, are lesions of the skin caused by chronic pressure on soft tissues. The most common victims of pressure ulcers are patients who are subject to prolonged immobility, advanced age, or, more frequently, patients who suffer from diseases that affect blood flow, e.g., diabetes. If left untreated, pressure ulcers will continue to grow until they become large enough to cause permanent muscle and nerve damage, and they may even become fatal.

Measuring sizes of pressure ulcers, and other types of wounds, can be helpful for a variety of reasons. For example, in order to appropriately stage a pressure ulcer and determine if it is improving or worsening, the pressure ulcer can be dimensioned and tracked over an extended period of time. For another example, measuring a size of a wound can help indicate what size wound dressing to use on the wound. However, it can be difficult to measure sizes of wounds.

Manual measurement of wounds can take an extended period of time while a person manually calculates wound size. Mathematical errors can inadvertently occur in such manual calculation, which can cause any number of undesirable effects, such as inaccurate data being recorded about and relied on for treatment of the wound. Manual measurements can vary by person taking the measurements and even by the same person taking measurements at different times, e.g., on different days. Such variance can lead to inconsistent data over time with data points that cannot be accurately compared to one another.

A wound can be sensitive to touch, especially within an interior of the wound that can extend relatively deeply into a body of a patient. Measuring a size of a wound can thus be uncomfortable or painful to a patient when a measurement device is located within the wound's interior.

A wound can have an irregular shape. A mathematical formula to manually calculate size, such as formulas for area and volume, may therefore be complicated and/or not easily determined for size calculation because the wound will not correspond to a traditional shape, such as circular, triangular, or ovular. Even if a mathematical formula is identified that can be manually processed, it can be difficult to determine appropriate values of variables such as length, width, and depth that are traditionally used in mathematical calculations since different areas of an irregularly sized wound can yield different values for those variables.

Accordingly, there remains a need for improved devices, systems, and methods for tissue and wound measurement.

### SUMMARY

The present invention generally provides devices, systems, and methods for tissue measurement.

In one aspect, a device is provided that in one embodiment includes a housing configured to be handheld, a sensor disposed within the housing, and an output device disposed within the housing. A distal end of the housing can be configured to trace a perimeter of a target tissue of a patient. The sensor disposed can be configured to collect coordinates at each of a plurality of discrete points around the perimeter when the distal end traces the perimeter. Each of the coordinates can be based on a position of the distal end relative to the target tissue at the discrete point at which the coordinates are collected. The output device can be configured to output at least one of the collected coordinates and a size of the target tissue that is based on the collected coordinates.

Each of the coordinates can include (x,y) dimensional data. The device can include a processor configured to calculate the size of the wound using the (x,y) dimensional data for each of the coordinates. The size of the wound can include at least one of a surface area of the enclosed by the perimeter and a length of the perimeter. Each of the coordinates can include (z) dimensional data, and the processor can be configured to calculate the length of the perimeter using the (x,y,z) dimensional data for each of the coordinates. The distal end can have indicia formed thereon. The target tissue can include a wound, and the indicia can be configured to provide (z) dimensional data for the wound when the distal end is inserted into the wound. The processor can be configured to calculate the size of the wound using the (z) dimensional data and using the (x,y) dimensional data for each of the coordinates. The size of the wound can include at least a volume of the wound.

The device can have any number of additional or alternative variations. For example, the distal end can have indicia formed thereon that can be configured to indicate a depth of the target tissue when the distal end is inserted into the target tissue. For another example, the output device can include a communication mechanism that is disposed within the housing. The communication mechanism can be configured to wirelessly communicate the at least one of the collected coordinates and the size of the target tissue to a computer that is separate and remote from the device. For yet another example, the output device can include at least one of a display screen and an audio speaker. The display screen can be visible on a surface of the housing, and the display screen can be configured to display the at least one of the collected coordinates and the size of the target tissue. The audio speaker can be configured to audibly provide the at least one of the collected coordinates and the size of the target tissue.

In another aspect, a system is provided that in one embodiment includes the device and a processor configured to calculate the size of the target tissue based on the collected coordinates.

In another aspect, a kit is provided that in one embodiment includes the device and a plurality of distal tips configured to be removably and replaceably attached to the housing. The housing can be configured to have one of the distal tips attached thereto at a time, and the distal end of the housing can be one of the distal tips.

In another embodiment, a device is provided that includes a housing configured to be handheld, a sensor disposed within the housing, and an output device disposed within the housing. A distal end of the housing can be configured to move along a surface of a target so as to make an invisible line along the surface. The sensor can be configured to collect coordinates at each of a plurality of discrete points along the invisible line when the distal end moves along the surface. Each of the coordinates can be based on a position of the distal end relative to the target at the discrete point at which the coordinates are collected. The output device can be configured to output at least one of the collected coordinates and a size characteristic of the target that is calculated from the collected coordinates.

In another aspect, a method is provided that in one embodiment includes gathering coordinate data by making an invisible perimeter line around a complete perimeter of a target tissue of a patient with a sterile distal end of a handheld device so as to collect a series of coordinates around the complete perimeter of the target tissue, calculating a size of the target tissue using the gathered coordinate data, and storing the size in an electronic memory.

The method can vary in any number of ways. For example, each of the coordinates in the series of coordinates can include (x,y) dimensional data of the target tissue. The calculating can use the (x,y) dimensional data for each of the coordinates, and the size can include at least one of a surface area of the target tissue and a length of the complete perimeter. For another example, each of the coordinates in the series of coordinates can include (x,y,z) dimensional data, the calculating can use the (x,y,z) dimensional data for each of the coordinates, and the size can include the length of the complete perimeter. For another example, the method can include measuring a depth of the target tissue. The calculating can also use the measured depth, and the size can include a volume of the target tissue. For still another example, the method can include removing the sterile distal end from a body of the handheld device, attaching a second sterile distal end to the body, gathering additional coordinate data by making a second invisible perimeter line around the complete perimeter of the target tissue with the second sterile distal end attached to the body, calculating a second size of the target tissue using the gathered additional coordinate data, and storing the second size in the electronic memory. For yet another example, the method can include displaying the size on at least one of a display screen that is on a body of the handheld device and on a display screen that is separate and remote from the handheld device. For another example, the method can include removing a portion of the handheld device that includes the electronic memory from a body of the handheld device. After the removal, the portion of the handheld device can be re-attached to the body of the handheld device. After the removal and before the re-attaching, the portion of the handheld device can be attached to a computer, thereby allowing the computer to access to stored size. For yet another example, the method can include wirelessly communicating the stored size from the electronic memory to a second electronic memory that is separate and remote from the handheld device. For still another example, storing the size can include wirelessly communicating the gathered size data to a computer that is separate and remote from the handheld device. For another example, the method can include, after a passage of time from gathering the coordinate data, gathering second coordinate data by making a second invisible perimeter line around the complete perimeter of the target tissue with at least one of the handheld device and a second sterile distal end of a second handheld device, calculating a second size of the target tissue using the gathered second coordinate data, and determining a change in a size of the target tissue between a time when the coordinate data was gathered and a time when the second coordinate data was gathered by comparing the size and the second size.

### BRIEF DESCRIPTION OF DRAWINGS

This invention will be more fully understood from the following detailed description taken in conjunction with the accompanying drawings, in which:

FIG. 1 is a schematic view of an embodiment of a network system;

FIG. 2 is a schematic view of an embodiment of a computer;

FIG. 3 is an exploded perspective view of an embodiment of a housing of a handheld device that includes a distal tip and a body;

FIG. 4 is an exploded perspective view of the body of FIG. 3 and components disposable therein;

FIG. 5 is a perspective view of an embodiment of a handheld device that includes a body and a distal tip;

FIG. 6 is another perspective view of the handheld device of FIG. 5;

FIG. 7 is a side view of the handheld device of FIG. 5;

FIG. 8 is another side view of the handheld device of FIG. 5;

FIG. 9 is a perspective view of the distal tip of FIG. 5 removed from the body;

FIG. 10 is another perspective view of the distal tip of FIG. 9;

FIG. 11 is a side view of the distal tip of FIG. 9;

FIG. 12 is a perspective view of another embodiment of a handheld device that includes a body and a distal tip;

FIG. 13 is an exploded side view of the handheld device of FIG. 12;

FIG. 14 is an exploded side view of another embodiment of a handheld device that includes a body and a distal tip;

FIG. 15 is a side view of a portion of the body of FIG. 14;

FIG. 16 is a side view of the distal tip of FIG. 14 and a portion of the body of FIG. 14;

FIG. 17 is a side view of another embodiment of a handheld device that includes a body and a distal tip, the device having a protective cover disposed over a distal portion thereof;

FIG. 18 is a perspective view of another embodiment of a distal tip for a handheld device; and

FIG. 19 is a top schematic view of a tissue wound.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Various devices, systems, and methods for tissue measurement are provided. In general, the devices, systems, and methods can allow a size of a tissue of a patient to be determined using a handheld device. In general, a handheld device can be configured to trace a perimeter of a target tissue, e.g., a wound, of a patient so as to allow a size of the target tissue to be determined. Examples of the size of the target tissue that can be determined include a length of a perimeter of the target tissue, a surface area of the target tissue, a depth of the target tissue, and a volume of the target tissue. The handheld device can include a housing configured to be handheld by a user with one hand, similar to the handling of a pen or a paintbrush. Tracing is a skill that requires minimal training or practice before being performed, particularly for adults, who would typically be the user handling the handheld device during the tracing. The handheld device can therefore be used to determine size with minimal training or practice, which can facilitate use of the handheld device. The tracing can be so simple that in some circumstances, the user of the handheld device need not even be a medical professional. This can allow, for example, patients to measure their own target tissues, e.g., wounds, when not at a medical care facility or otherwise with a medical professional. The patient's condition can thus be more frequently monitored and evaluated, and the patient need not visit a medical care facility in order for their care provider to receive current information.

The handheld device can be configured to gather data regarding target tissue size and to communicate the data it gathers to a separate and remote device, such as a computer, which can facilitate medical professional access to the gathered data, even when the patient is remote from the medical professional, and/or can facilitate inclusion of the gathered data in a patient's medical record. The gathered data can include dimensional data in two dimensions, e.g., in x and y dimensions, or in three dimensions, e.g., in x, y, and z dimensions. The handheld device can thus be configured to determine two-dimensional (2D) size (e.g., length, surface area, etc.) using gathered 2D data and to determine three-dimensional (3D) size (e.g., volume, etc.) using gathered 3D data. The handheld device can be configured to store the determined target tissue size, which can facilitate comparison of the data with previous and/or future size data of the target tissue. The handheld device can be configured to determine the size of the target tissue using an on-board processor, and/or a processor separate and remote from the handheld device can be configured to determine the size of the target tissue using data gathered by the handheld device. Using an on-board processor can allow the handheld device to function as a convenient standalone unit and/or to provide the determined size when a user operating the handheld device is present with the patient, which can facilitate quick evaluation and/or sharing of information. Using a separate and remote processor can allow for more processing capabilities than may be available in the relatively small handheld device and/or can help lower a cost of the handheld device by it not including an on-board processor. The handheld device can be configured to gather any one or more types of patient data in addition to data used to determine target tissue size, such as any one or more of temperature, pH, biological or chemical composition (e.g., presence of enzymes, matrix metalloproteinases, or other proteins), presence of exudates, composition of exudate (e.g., color, composition, and viscosity) moisture (e.g., water) content, texture (e.g., hardness and profile), color/tint, and turgor (e.g., tissue elasticity), which can streamline patient evaluation and treatment.

The handheld device can be configured to be reusable on the same patient and/or on different patients, which can facilitate cost-effectiveness of the device. The handheld device can be configured to be reusable by being disposable in its entirety or by having a removable distal tip that can be reattached to the handheld device, e.g., after being cleaned, and/or that can be replaced on the handheld device with another distal tip, e.g., a new, unused distal tip.

The handheld device can be configured to directly contact tissue of the patient while tracing the target tissue's perimeter. The handheld device directly contacting the patient's tissue can allow the target tissue's size to be measured without the patient having to be specially prepared for the target tissue measurement, such as by any measurement tools (e.g., rulers, transparent films, etc.), other than the handheld device, being placed in contact with the target tissue and/or other portion of the patient, and/or by requiring additional equipment such as a display screen, a pencil or other writing instrument, a ruler, a calculator, etc. to be available to aid in the measuring. The handheld device need not, however, directly contact the patient's tissue in order to gather data, which can help prevent the tissue from being irritated, damaged, etc. from direct contact and can help reduce chances of patient discomfort.

The handheld device can have an on-board power supply such that the device need not be plugged into a power source, such as an electrical outlet, for the tracing to be performed. The handheld device can thus be easily portable and/or be unencumbered by cords, wires, etc. during use.

Examples of a wound that can be measured using the devices, systems, and methods disclosed herein include tissue wounds such as a venous ulcer, a diabetic ulcer, a pressure ulcer, a post-surgical skin wound, a skin lesion, a blister, and a burn. In an exemplary embodiment, a handheld device can be used to measure a tissue wound on an exterior surface of a patient's skin. A handheld device can, however, be used to measure a tissue wound within a patient, such as on an exterior surface of an internal organ.

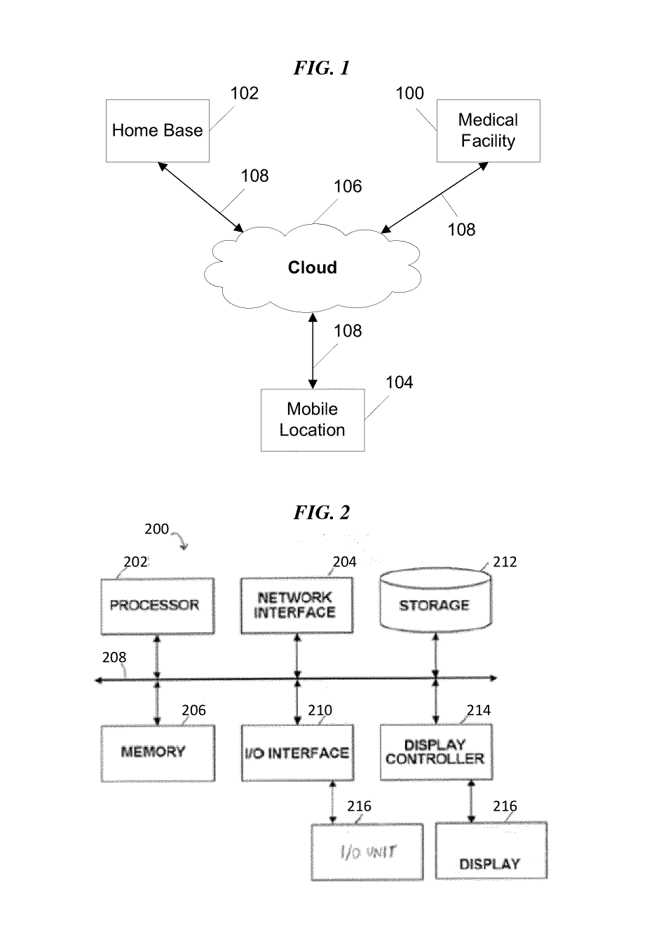

Any of a variety of users can use a handheld device as disclosed herein in any of a variety of locations. For example, as shown in an embodiment illustrated in FIG. 1, a handheld device (not shown) can be used in any number of locations such as a medical facility 100 (e.g., a hospital, an operating room, a nurse's station, a medical device company, a doctor's office, an outpatient or physical therapy clinic, etc.), a home base 102 (e.g., a patient's home, a patient's workplace, etc.), a mobile location 104 (e.g., at a site of an accident, etc.), and so forth. Examples of users who can use the handheld device include patients having wounds to be measured, medical professionals (e.g., surgical technicians, doctors, nurses, medical assistants, home health aides, physical therapists, etc.), significant others of patients, friends of patients, and family members of patients.

The handheld device can be configured to access a network 106 through a wired and/or wireless connection 108. FIG. 1 shows the network 106 as a cloud, but the network can be in this and/or other forms, e.g., a local area network (LAN), a wide area network (WAN), the Internet, etc., as will be appreciated by a person skilled in the art. In an exemplary embodiment, the handheld device can be configured to access the network 106 wirelessly, e.g., through wireless connection(s) 108 (e.g., WiFi, Bluetooth, etc.), which can facilitate usability of the handheld device from almost any location in the world. Data can be transmitted wirelessly using an existing protocol, such as 802.11, or a proprietary protocol, e.g., a protocol that optimizes power, data, and range for a particular use more than an existing protocol.

The handheld device can be configured to access the network 106 using one or more security features such that various aspects of the network 106 available to any particular user can be determined based on the identity of the user and/or the location from which the user is accessing the network 106. To that end, each user can have a unique username, password, and/or other security credentials to facilitate access to the network 106. The handheld device can be configured to allow manual entry of the security credential(s), such as by including a touchscreen thereon configured to receive touch input from the user, and/or can be preprogrammed with security credential(s), such as a unique identification code of the handheld device, that can be communicated to the network 106. The received security parameter information can be checked against a database of authorized users to determine whether the user is authorized and to what extent the user is permitted to interact with the network 106, view stored information, and so forth.

The devices, systems, and methods disclosed herein can be implemented using one or more computers. As discussed further below, an exemplary embodiment of a handheld device can include an on-board computer that includes at least a processor, a memory, and a display.

The term “computer” as used herein refers to any of a variety of digital data processing devices, e.g., laptop or notebook computers, tablet computers, slate computers, server computers, graphics tablets, interactive whiteboards, mobile phones, personal digital assistants (PDAs), gaming systems, televisions, radios, portable music players, and the like. The terms “display” and “display screen” as used herein refers to any of a variety of display devices, e.g., a liquid crystal display (LCD), a light-emitting diode (LED) screen, a cathode ray tube (CRT) screen, a touch screen, a 3D screen, and the like. Additionally, the term terms “display” and “display screen” as used herein can refer to a display that is fixedly mounted in the same chassis or package as a base of an electronic device, as well as to displays that are removably and replaceably connected, wired or wirelessly, to the same chassis or package as a base of an electronic device.

A computer can include and/or be in communication with any of a variety of software and/or hardware components. In addition, although an exemplary computer is depicted and described herein, a person skilled in the art will appreciate that this is for sake of generality and convenience. In other embodiments, the computer may differ in architecture and operation from that shown and described with respect to any of the illustrated embodiments.

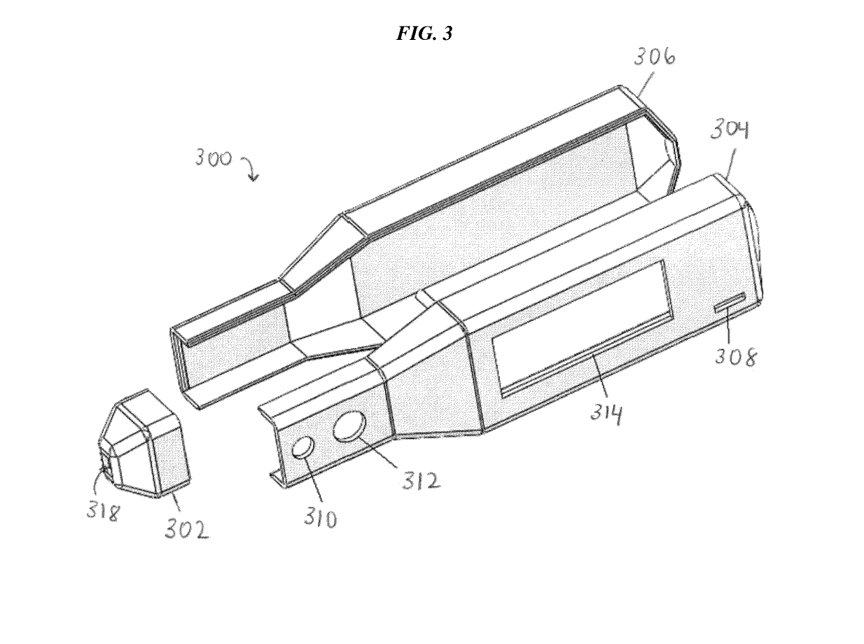

As shown in FIG. 2, a computer 200 can include a processor 202 which controls the operation of the computer 200, for example by executing an operating system (OS), a basic input/output system (BIOS), device drivers, application programs, and so forth. The processor 202 can include any type of microprocessor or central processing unit (CPU), including programmable general-purpose or special-purpose microprocessors and/or any one of a variety of proprietary or commercially-available single or multi-processor systems. The computer 200 also includes a memory 206, which can provide temporary storage for code to be executed by the processor 202 or for data that is processed by the processor 202. The memory 206 can include read only memory (ROM), flash memory, one or more varieties of random access memory (RAM), and/or a combination of memory technologies. The various elements of the computer 200 can be coupled to a bus system 208. The illustrated bus system 208 is an abstraction that a person skilled in the art will appreciate represents any one or more separate physical busses, communication lines/interfaces, and/or multi-drop or point-to-point connections, connected by appropriate bridges, adapters, and/or controllers.

The computer 200 can also include a network interface 204, an input/output (I/O) interface 210, a storage device 212, and a display controller 214. The network interface 204 can enable the computer 200 to communicate with remote devices, e.g., other computers, over a network. The I/O interface 210 can facilitate communication between one or more I/O units 216. A person skilled in the art will appreciate that the computer 200 can be configured to communicate with a variety of I/O units 216. Examples of input units include a keyboard, a mouse, a joystick, and a pointing device. Examples of output units includes a speaker, a printer, a scanner, a removable memory, and the various other components of the computer 200. The storage device 212 can include any conventional medium for storing data in a non-volatile and/or non-transient manner. The storage device 212 can thus hold data and/or instructions in a persistent state, i.e., the value is retained despite interruption of power to the computer 200. The storage device 212 can include one or more hard disk drives, flash drives, universal serial bus (USB) drives, optical drives, various media disks or cards, and/or any combination thereof, and can be directly connected to the other components of the computer 200 or remotely connected thereto, such as over a network. The display controller 214 can include a video processor and a video memory, and can generate images to be displayed on a display 216 in accordance with instructions received from the processor 202.

One or more software modules can be executed by the computer 200 to facilitate human interaction with the computer 200. These software modules can be part of a single program or one or more separate programs, and can be implemented in a variety of contexts, e.g., as part of an operating system, a device driver, a standalone application, and/or combinations thereof. A person skilled in the art will appreciate that any software functions being performed by a particular software module can also be performed by any other module or combination of modules.

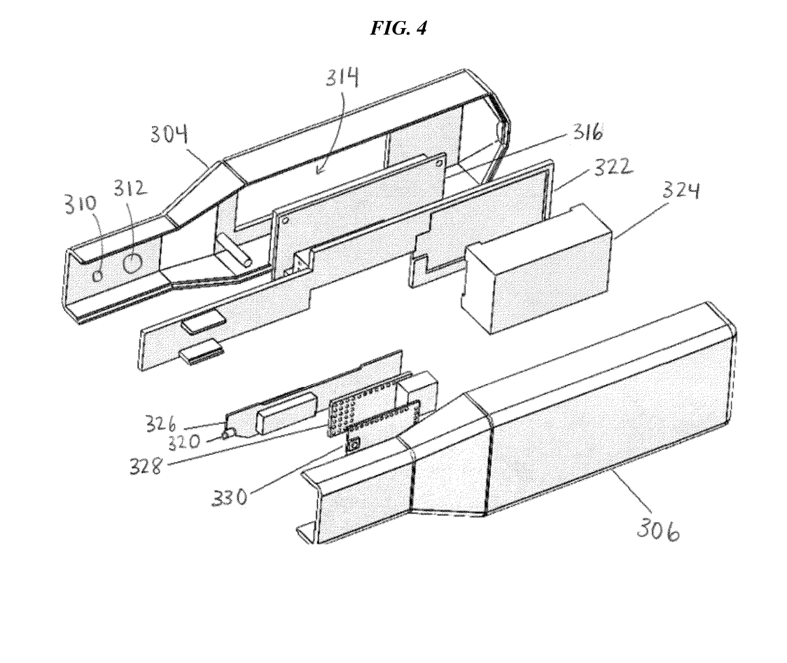

FIGS. 3 and 4 illustrate an embodiment of a handheld device configured to trace a perimeter of a wound of a patient so as to allow a size of the wound to be determined. As shown in FIG. 3, the handheld device can include a housing 300 configured to be handheld by a user. The housing 300 can be fluid-tight, which can help prevent components disposed within the housing 300 from being exposed to potentially damaging fluid such as water, blood, saline, etc. The housing 300 can be a singular piece, or, as in the illustrated embodiment, can include a plurality of components that are attached together to form the housing 300. The housing 300 can include any plural number of components. In the illustrated embodiment, the housing 300 includes a distal tip 302 and a body that includes a first body portion 304 and a second body portion 306. The distal tip 302 and the body portions 304, 306 can each have a variety of sizes, shapes, and configurations.

The housing 300 is shown in FIG. 3 with the distal tip 302 and the body portions 304, 306 disassembled and in FIG. 4 with the first and second body portions 304, 306 disassembled (the distal tip 302 is not shown in FIG. 4). In an exemplary embodiment, at least a portion of the housing 300 that includes computer component(s) disposed therein can be permanently attached together, e.g., permanently assembled together during manufacturing, which can help protect the component(s) from being damaged. All computer components of this illustrated embodiment are disposed within a body of the device that includes the first and second body portions 304, 306, as shown in FIG. 4 and as discussed further below. The first and second body portions 304, 306 can thus be permanently attached together. The distal tip 302 can be removably and replaceably attached to the body, as discussed further below.

The housing 300 can be fluid tight when all of the distal tip 302 and the body portions 304, 306 are assembled together. At least a portion of the housing 300 can be fluid tight even when one or more of its components are removed therefrom. For example, the portion of the housing 300 that includes computer component(s) therein can be fluid tight even when a remainder of the housing 300 is removed therefrom. As in the illustrated embodiment, the body of the housing 300 that includes the first and second body portions 304, 306 can have the computer component(s) disposed therein and can be fluid tight even when the distal tip 302 is removed therefrom.

The housing 300 can be made from a variety of materials. In an exemplary embodiment, the housing 300 can be made from one or more biocompatible materials, e.g., stainless steel, polyethylene, etc. The material(s) can be rigid, which can help protect the components disposed within the housing 300. In an exemplary embodiment, the first and second body portions 304, 306 can be made from the same material(s), while the distal tip 302 can be formed from the same or different material(s) than the body portions 304, 306. The distal tip 302 can be made from a less expensive material than the body portions 304, 306, e.g., a plastic instead of a metal, which can facilitate disposability of the distal tip 302.

As shown in FIG. 3, the body of the handheld device can include a power button 308, a reset button 310, a read button 312, and a display window 314. The buttons 308, 310, 312 are all depressible push buttons in the illustrated embodiment, but any one or more of the buttons 308, 310, 312 can have other configurations that are configured to allow actuation by a user, e.g., a switch, a lever, etc. Additionally, the locations of the buttons 308, 310, 312 and the display window 314 on the first body portion 304 are illustrative examples. Any of the buttons 308, 310, 312 and the display window 314 can be positioned elsewhere on the body. The power button 308 can be configured to be actuated by a user, e.g., pushed, to selectively turn the handheld device on or off. The reset button 310 can be configured to be actuated by a user, e.g., pushed, when the device is “on” to reset the handheld device so as to ready the device for a new measurement reading. The read button 312 can be configured be actuated by a user, e.g., pushed, to start and stop measurement readings when the device is “on.”

The display window 314 is rectangular in the illustrated embodiment, but the display window 314 can have any of a variety of other sizes. The display window 314 can include a cut-out in a sidewall of the device's body, e.g., in the first body portion 304, in which a display screen 316 can be positioned so as to be viewable by a user. The display window 314 can be any size. In the illustrated embodiment, the display window 314 is sized to accommodate the display screen 316 that in this embodiment includes a 16×2 character LCD. The display screen 316 can, however, have other sizes and can be a type other than LCD. The display screen 316 can be entirely disposed within the housing 300, as in the illustrated embodiment.

In an exemplary embodiment, the buttons 308, 310, 312 and the display window 314 can all be on a same side of the handheld device, e.g., all on the first body portion 304, which can facilitate confirmation of actuation of the buttons 308, 310, 312 by displaying an indication of their actuation on the screen 316. For example, when the power button 308 is actuated, the screen 316 can illuminate. For another example, when the read button 312 is actuated, the screen 316 can display text indicating that measurement is in progress. The display window 314 can be an open space cut-out in the body, or the display window 314 can have a transparent protective cover disposed therein, such as a transparent plastic film, which can help protect the screen 316.

The distal tip 302 can be attached to a distal end of the body, e.g., to distal ends of the first and second body portions 304, 306. In this illustrated embodiment, the distal tip 302 is non-removably attached to the body. The distal tip 302 can, however, be configured to be removably and replaceably attachable to the body.

The distal tip 302 can be sterile, which can facilitate safe use of the distal tip 302 near and/or in direct contact with tissue of a patient. The distal tip 302 can be a hollow member, which can facilitate visualization therethrough, as discussed further below. The distal tip 302 can be distally tapering along at least a partial longitudinal length thereof. In the illustrated embodiment, a distal portion of the distal tip 302 distally tapers. By distally tapering, the distal tip 302 can be more accurately positioned adjacent a target tissue, e.g., a wound, a surface of an internal organ, a skin surface, etc., and/or can be better able to fit within a small space. A distal end of the distal tip 302 can be substantially flat, as in this illustrated embodiment. Being substantially flat can facilitate smooth tracing of the distal tip 302 along a surface while minimizing chances of the distal tip 302 damaging the surface. A person skilled in the art will appreciate that the distal tip's distal end being substantially flat can include a precisely flat surface or a surface that is not precisely flat because of insignificant variances, e.g., tolerances allowed in manufacturing.

The distal tip 302 can have a viewing window 318 formed therein. The viewing window 318 can be at the distal end of the distal tip 302, similar to the location of a ballpoint pen's ballpoint. The viewing window 318 can be an open space cut-out in the distal tip 302, or the viewing window 318 can have a transparent protective cover disposed therein, which can help prevent fluid and/or other material from entering the distal tip 302 therethrough. The viewing window 318 has a square shape in this illustrated embodiment, but the viewing window 318 can have other shapes. The viewing window 318 can have a variety of sizes. The viewing window 318 should have a size and shape configured to allow visualization therethrough for a viewing element 320 disposed in the housing 300, as discussed further below.

A proximal end (obscured in FIG. 3) of the distal tip 302 can be open such that the distal tip 302 is similar to a cup. Alternatively, the proximal end of the distal tip 302 can have a window formed therein, similar to the viewing window 318, through which the viewing element 320 can visualize. The proximal window can be longitudinally aligned with the viewing window 318 to facilitate the viewing element's visualization through the proximal window and the viewing window 318. The proximal window can be open or can have a transparent protective cover disposed therein.

As shown in FIG. 4, a plurality of components can be disposed within the housing 300. The plurality of components can include the display screen 316, an electronics mount 322, a power source 324, an optical navigation chip 326 coupled to the viewing element 320, a storage unit such as a universal serial bus (USB) host shield 328, and a processor 330. Other components (not shown) that can be disposed in the housing 300 include a wireless communication mechanism, a bus, a gyroscope, an inertial measurement unit (IMU), a magnetic compass, an image gathering device (e.g., a still image camera, a video image camera, etc.) and a sensor (e.g., for temperature, color, biologic or chemical composition, pH, etc.).

The electronics mount 322 can be configured to help securely position electronics within the housing 300 so as to help prevent them from being jostled and damaged. The electronics mount 322 can be configured to seat the display screen 316, the power source 324, the optical navigation chip 326, the USB host shield 328, and the processor 330 on various surfaces thereof.

The power source 324 can be configured as an on-board power supply for the handheld device's various electronics components. The power source 324 in the illustrated embodiment includes one 9 V battery, but the power source 324 can include any number of individual power sources, can be another size and/or type of battery (e.g., AAA size, lithium type, etc.), and can include one or more power sources other than a battery (e.g., solar cells). The power source 324 can be configured to be removably and replaceably attached to the housing 300, which can facilitate replacement of a depleted power source. In the illustrated embodiment, the power source 324 is non-removably disposed within the housing 300, which as mentioned above, has its body portions 304, 306 permanently attached together.

The optical navigation chip 326 can be configured to gather optical data. The optical navigation chip 326 can include any number and any type of optical navigation electronics, as will be appreciated by a person skilled in the art, such as LEDs, photodiodes, photoresistors, etc. The viewing element 318, e.g., an LED, can be optical navigation electronics attached to the optical navigation chip 326 and can be configured to provide an optical signal in a direction of the distal tip 302. The viewing element 318 can be longitudinally aligned with the viewing window 318 and the distal tip's proximal window so as to facilitate direction of the optical signal to a target that is adjacent to the viewing window 318. As discussed further below, the optical data gathered by the optical navigation chip 326 can be used to determine wound size. As also discussed further below, the optical data gathered by the optical navigation chip 326 can be stored in the memory (e.g., the USB), processed by the processor 330, wirelessly communicated via the wireless communication mechanism, and/or transmitted via the USB host shield 328. In general, the optical navigation chip 326 can be configured to gather optical data when the distal tip 302 is tracing a perimeter of wound or other item, and the gathered optical data can be processed by the processor 330, and/or an external processor (not shown), so as to determine a size of the wound or other item.

Optical data is one example of data that a handheld device can gather to facilitate wound measurement determinations. Thus, although the handheld device in this illustrated embodiment is configured to gather optical data using the optical navigation chip 326, another handheld device can be configured to gather another type of data instead of or in addition to optical data. For example, a handheld device can be configured to gather high definition (HD) imaging data using, e.g., an HD camera disposed within the device's housing. The HD imaging data can be analyzed by, e.g., a processor executing imaging software that analyzes the gathered images so, for example, determine a size of a wound shown in the images. In an exemplary embodiment, the gathered HD images can be in color. For another example, a handheld device can be configured to gather 3D profiling data. The 3D profiling data can be analyzed by, e.g., a processor executing profiling software that analyzes gathered 3D surface data so as to, for example, determine a size of a wound represented in the 3D profiling data. For yet another example, a handheld device can be configured to gather laser navigation data, which can be similar to gathering optical navigation data except that a laser, e.g., a laser diode, can be used to gather laser data instead of an optical element such as a photodiode being used to gather optical data.

The handheld device can include multiple mechanisms configured to gather data to be analyzed for size determination. The processor 330 and/or an off-board processor can be configured to analyze the data gathered by each of these mechanisms, which can help provide a more accurate size measurement. For example, the handheld device can include the optical navigation chip 326 and the viewing element 320 that gather optical data and can include a camera that gathers still and/or video image data. In an exemplary embodiment, the gathered images can be in color.

The handheld device can include one or more angular measurement mechanisms configured to facilitate normalization of measurements gathered by the device. A user will typically hold the device at different angular orientations relative to the target, e.g., for hand comfort reasons, as the device is moved relative to the target. The angular measurement mechanism(s) can be configured to gather data when the device is tracing a target so as to allow for angular orientation or tilt of the device relative to the target to be compensated for with respect to data gathered via the viewing element 320. Examples of angular measurement mechanisms include a gyroscope, an IMU, and a magnetic compass.

The USB host shield 328 can be configured to facilitate temporary connection of the handheld device to another USB-enabled device, such as a USB flash drive, a USB data communication cable, etc. The USB host shield 328 can include a USB peripheral/host controller with digital logic and analog circuitry that facilitate USB connection. The handheld device can, however, include a type of USB connector other than a USB host shield, which can be particularly useful when the processor 330 is an Arduino processor. The USB host shield 328 can be configured to facilitate transfer of data gathered by the optical navigation chip 326, data stored in the memory, and/or data analyzed by the processor 330 to an external device. The USB host shield 328 can also be configured to facilitate receipt of external data to be stored in the memory and/or analyzed by the processor 330, such as data regarding a specific patient that the handheld device will be or has been used on, e.g., patient identification data, patient health record data, previous wound measurements for the patient, etc.

The processor 330 can be configured to control various elements of the handheld device, as will be appreciated by a person skilled in the art. The processor 330 can be configured to process data gathered by the optical navigation chip 326 (and/or other data gathering mechanism). For example, the processor 330 can be configured to analyze coordinate data gathered by the optical navigation chip 326 (and/or other data gathering mechanism) so as to determine a size of a wound, as discussed further below. The processor 330 in the illustrated embodiment includes an Arduino Pro Mini, but a handheld device can include another Arduino processor or any other type of processor.

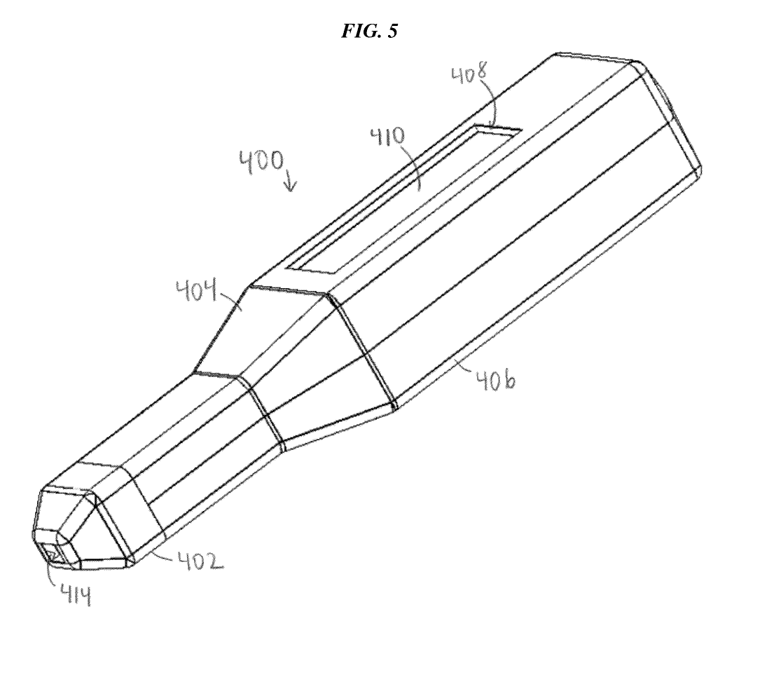

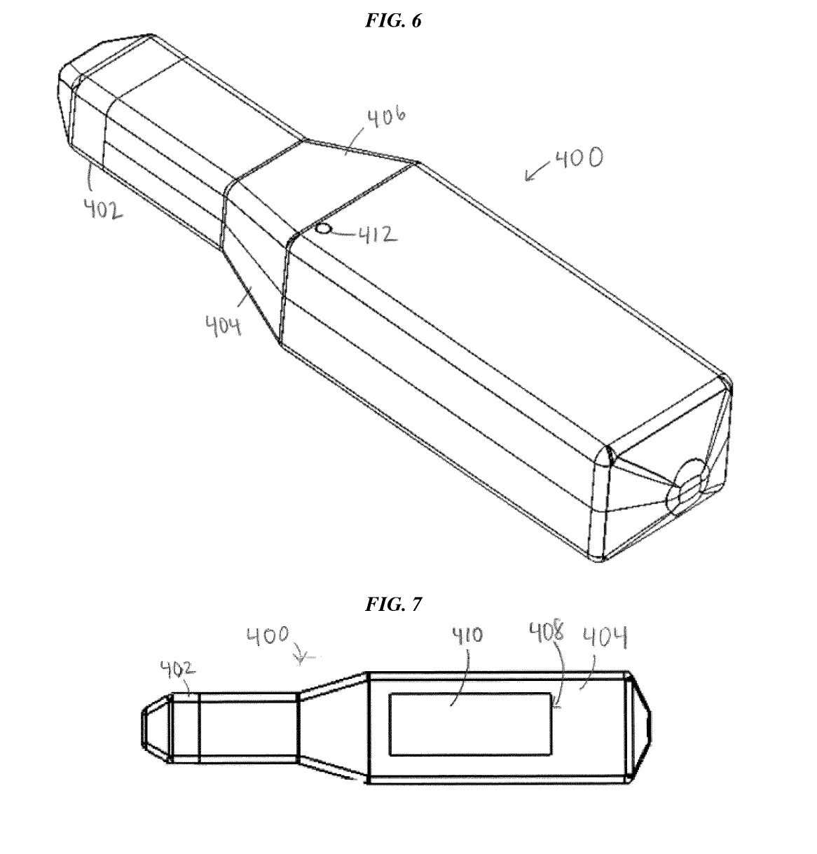

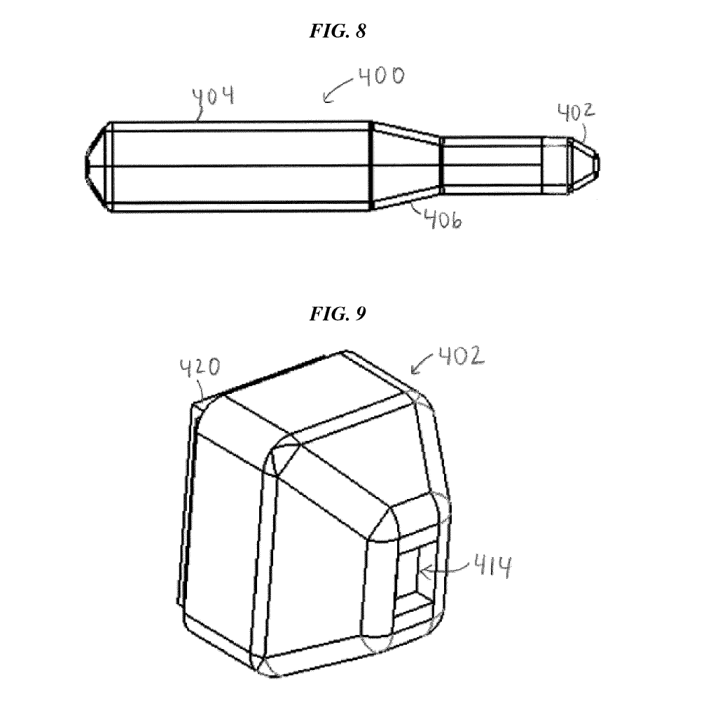

FIGS. 5-8 illustrate another embodiment of a handheld device 400 configured to trace a perimeter of a wound of a patient so as to allow a size of the wound to be determined. The handheld device 400 can include a housing that includes a body and a distal tip 402 coupled to the body. The body can include first and second body portions 404, 406, which in this illustrated embodiment are permanently attached together. The body, e.g., the first body portion 404, can include a display window 408 through which a display screen 410 disposed within the housing can be visible. The body, e.g., the first body portion 404, can include a power button 412 (see FIG. 6).

The distal tip 402, also shown in FIGS. 9-11, in this illustrated embodiment includes a distal portion that distally tapers, includes a substantially flat distal end, includes a viewing window 414 (see FIGS. 5 and 9), and includes a proximal window 416 (see FIG. 10). The distal tip 402 can be configured to be removably and replaceably attached to the body, as in this illustrated embodiment.

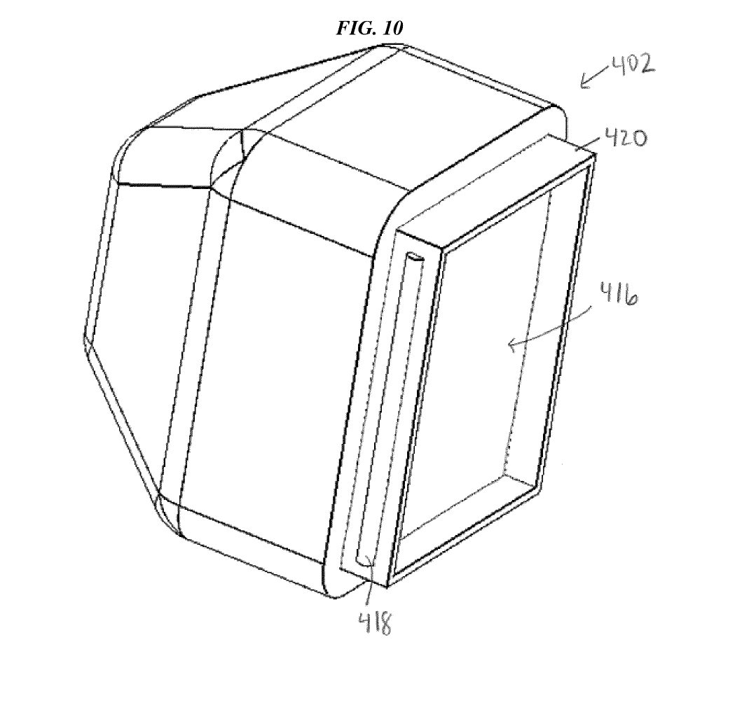

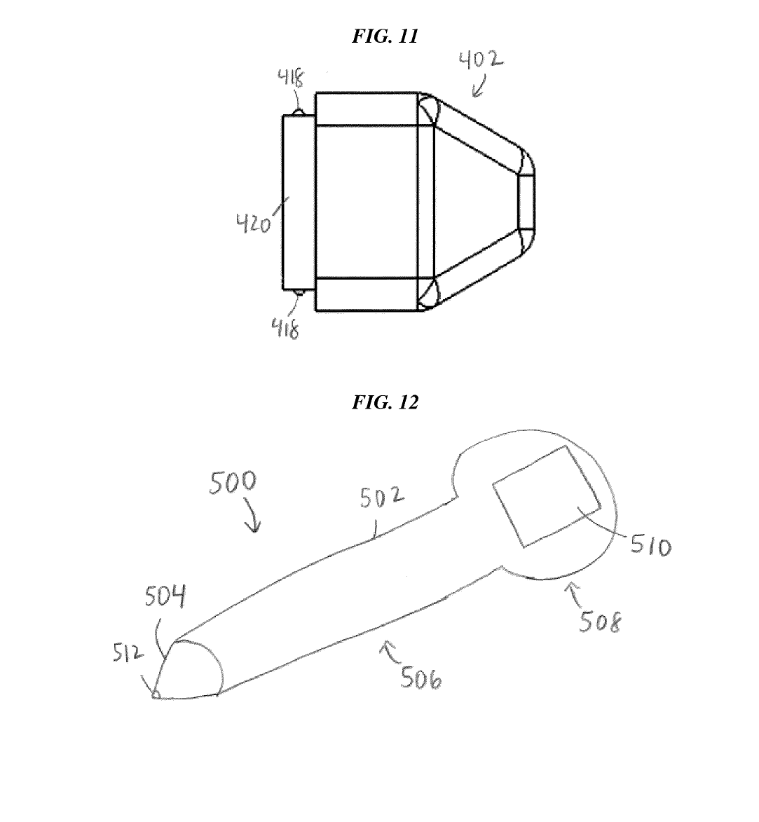

The distal tip 402 can be removably and replaceably attached to the body in a variety of ways. As in the illustrated embodiment, a proximal end of the distal tip 402 can be configured to releasably mate with a distal end of the body, e.g., to distal ends of the body portions 404, 406. The distal tip's proximal end can include an attachment mechanism configured to mate with a corresponding attachment mechanism of the device's body. For example, the distal tip's attachment mechanism can include a male or female component such as a thread configured to threadably mate with a corresponding male or female component such as a thread formed on or in the body. For another example, the distal tip's attachment mechanism can include a clip configured to clip onto a surface of the device's body. For yet another example, the distal tip's attachment mechanism can include a rail configured to slidably mate with a track formed in the device's body. Similarly, the distal tip's attachment mechanism can include a track configured to slidably mate with a rail formed on the device's body. For another example, as in the illustrated embodiment, the distal tip's attachment mechanism can include a snap fit mechanism. The distal tip 402 can include a protrusion 418 configured to facilitate snapping of the distal tip 402 onto the device's body. The distal tip 402 includes two protrusions 418 in this illustrated embodiment, but a distal tip can include any number of protrusions. Similarly, the two protrusions 418 in this illustrated embodiment are located on opposite sides of the distal tip 402, but protrusions can be located elsewhere. Although the distal tip 402 includes the protrusion 418, the body can instead include the protrusion. The body can include a depression (not shown) configured to seat the protrusion 418. As in the illustrated embodiment, the protrusion 418 can be configured to be seated within a distal lip (not shown) of the body so as to snap the distal tip 402 to the body. The distal tip 402 can include a proximal lip 420 configured to be inserted into the body so as to facilitate attachment of the distal tip 402 to the body. The proximal lip 420 can include a reduced diameter region of the distal tip 402. The protrusion 418 can be formed on the proximal lip 420, as shown in FIGS. 10 and 11. The protrusion 418 can thus be configured to be located within the body when the distal tip 402 is attached thereto, as shown in FIGS. 5-8.

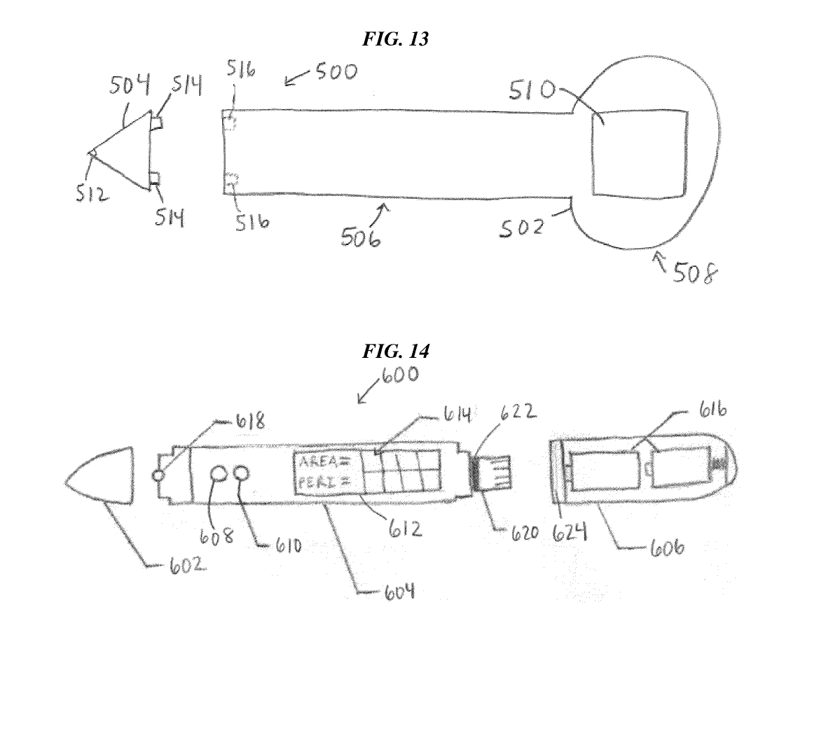

FIGS. 12 and 13 illustrate another embodiment of a handheld device 500 configured to trace a perimeter of a wound of a patient so as to allow a size of the wound to be determined. The handheld device 500 can include a housing that includes a body 502 and a distal tip 504 coupled to the body 502. The body 502 in this illustrated embodiment includes a singular element. The body 502 can include a shaft portion 506 and a display portion 508. The shaft portion 506 can be configured to be handheld by a user, similar to a shaft of a pen. The display portion 508 can also be configured to be handheld by a user, but the shaft portion 506 can be configured to be more naturally gripped and held than the display portion 508, which can be visible to the user when holding the shaft portion 506. The shaft portion 506 can be cylindrical, as in the illustrated embodiment, though the shaft portion 506 can have other shapes, e.g., a rectangular box shape, a triangular prism shape, etc. The display portion 508 can include a display screen 510. The display portion 508 can be substantially flat, as in this illustrated embodiment.

The distal tip 504 in this illustrated embodiment includes a distal portion that distally tapers, includes a substantially pointed distal end, and includes a viewing window 512. The viewing window 512 in this illustrated embodiment includes a transparent distal portion of the distal tip 504, e.g., a transparent distal-most end thereof.

The distal tip 504 can be removably and replaceably attached to the body 502, as in this illustrated embodiment. A proximal end of the distal tip 504 can be configured to be removably and replaceably attachable to a distal end of the body 502. The distal tip 504 and the body 502 in this illustrated embodiment are configured to attach together via a snap fit mechanism. The distal tip 504 can include a mating element, e.g., a protrusion 514, and the body 502 can include a corresponding mating feature, e.g., a depression 516, configured to releasably mate with the mating element. The distal tip 504 includes two protrusions 514 extending proximally therefrom, and the body 502 includes two depressions 516 formed in a distal end thereof, but the device 500 can include any number of protrusions 514 and depressions 516. In an exemplary embodiment, a number of the protrusions 514 and the depressions 516 can be equal. When the device includes a plurality of protrusions 514 and a plurality of depressions 516, the protrusions 514 and the depressions 516 can be keyed such that each of the protrusions 514 can only be securely snap fit into a unique one of the depressions 516. Such keying can help ensure that the distal tip 504 is attached to the body 502 in a desired orientation, e.g., an orientation that aligns the viewing window 512 with a data gathering element (not shown) disposed within the body 502. In another embodiment, a distal tip can include depression(s) and a body can include protrusion(s) configured to be seated via snap fit in the depression(s).

FIG. 14 illustrates another embodiment of a handheld device 600 configured to trace a perimeter of a wound of a patient so as to allow a size of the wound to be determined. The handheld device 600 can include a housing that includes a body and a distal tip 602 coupled to the body. The body can include first and second body portions 604, 606, which in this illustrated embodiment are configured to be removably and replaceably attached together.

A plurality of components can be disposed within the housing. At least one of the components can be disposed within the first body portion 604 of the housing, and at least one of the components can be disposed within the second body portion 606 of the housing. In this way, the component(s) disposed within one of the body portions 604, 606 can be replaced without requiring replacement of the component(s) disposed in the other of the body portions 604, 606. Thus, less expensive components and/or components that can wear out more quickly than other components can be replaced without requiring expensive and/or unnecessary replacement of components that are still functioning and/or otherwise are not desired to be replaced.

The body of the handheld device 600 can include a power button 608, a reset button (not shown), a read button 610, and a display window 612. The buttons 608, 610 are each depressible push buttons in the illustrated embodiment, but any one or more of the buttons 608, 610 can have another configurations that are configured to allow actuation by a user.

The plurality of components disposed within the housing can include the display screen 614, a power source 616, a viewing element 618, and a storage unit such as a USB stick 620. Other components (not shown) that can be disposed in the housing include a wireless communication mechanism, a bus, a gyroscope, a sensor, an electronics mount, and a control unit (e.g., an optical navigation chip, etc.) for the viewing element 618. The display screen 614 in this illustrated embodiment is configured to show wound size measurements of total surface area and perimeter length, e.g., as numerical values, but as mentioned above, these and/or other size measurements can be provided by the handheld device 600.

The viewing element 618 can include, e.g., an LED 634, a lens 636, and a navigation sensor 638. In general, the LED 634 can be configured to shine a light directed distally, e.g., toward a target such as a wound perimeter, through the lens 636. The light can be reflected proximally through lens 636 and to the navigation sensor 638. The navigation sensor can include a digital camera such as a complementary metal oxide semiconductor (CMOS) sensor and a digital signal processor (DSP). The CMOS sensor can be configured to gather a plurality of images per second, e.g., 1500 images per second, and transmit the images to the DSP. The DSP can be configured to detect patterns in the target and determine whether the device, e.g., whether the distal tip 602, if moving based on the change in pattern.

The first body portion 604 can include the power button 608, the read button 610, the display window 612, the display screen 614, the viewing element 618, and the USB stick 620. The second body portion 606 can include the power source 616. The power source 616 can thus be configured to be replaced as needed, e.g., when the power source 616 nears depletion or is depleted, without requiring replacement of elements of the first body portion 604. The power source 616 in this illustrated embodiment includes two alkaline batteries, but as discussed above, the power source 616 can include any number and any type of power sources.

The first and second body portions 604, 606 can be configured to be removably and replaceably attached to one another such that the first and second body portions 604, 606 can be selectively detached from and reattached to one another any number of times. Alternatively, instead of being reattached to one another, a different second body portion, e.g., one with a new power source, can be attached to the first body portion 604, or a different first body portion, e.g., one with a new storage unit or one with a processor having increased processing capabilities, can be attached to the second body portion 606.

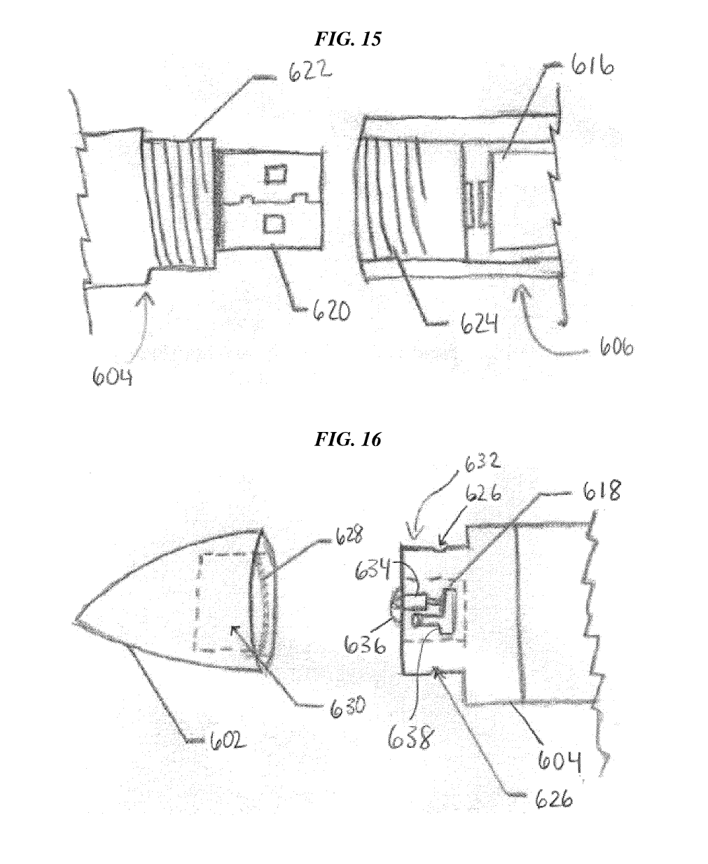

The first and second body portions 604, 606 can be configured to be removably and replaceably attached to one another in a variety of ways. As in the illustrated embodiment, a proximal end of the first body portion 604 can be configured to releasably mate with a distal end of the second body portion 606. The first body portion's proximal end can include an attachment mechanism configured to mate with a corresponding attachment mechanism of the second body portion 606. Examples of the attachment mechanisms include those discussed above with respect to the distal tip 402 releasably mating to the body of the device 400 of FIG. 5. In this illustrated embodiment, as shown in FIGS. 14 and 15, the first body portion's attachment mechanism includes a male mating component in the form of a thread 622, and the second body portion's attachment mechanism includes a female mating component in the form of a thread 624.

The USB stick 620 can be configured to be disposed at least partially within the second body portion 606 when the first and second body portions 604, 606 are attached together. The second body portion 606 can thus help protect the USB stick 620 from damage when the first and second body portions 604, 606 are attached together while allowing the USB stick 620 to be at least partially exposed and accessible to a user when the first and second body portions 604, 606 are not attached together. By being at least partially exposed and accessible to a user when the first and second body portions 604, 606 are not attached together, the USB stick 620 can be configured to be attached to a USB compatible component, e.g., a USB cable, a USB port, etc., so as to facilitate transmission of data from the handheld device to an external device and to facilitate transmission of data from an external device to the handheld device. In this way, the first body portion 604 can be used as a USB drive, which can facilitate easy sharing of data since USB drives are familiar devices that are easy to use for most users.

The distal tip 602 can have a variety of sizes, shapes, and configurations. At least a portion of the distal tip 602 can be substantially transparent so as to facilitate visualization of the viewing element 618 therethrough. In an exemplary embodiment, at least a distal-most portion of the distal tip 602 can be substantially transparent. The substantially transparent distal-most portion can be longitudinally aligned with the viewing element 618.

The distal tip 602 can be distally tapering along at least a partial longitudinal length thereof. In the illustrated embodiment, the distal tip 602 distally tapers along an entire longitudinal length thereof.

The distal tip 602 in this illustrated embodiment has a pointed distal-most tip. The pointed tip can simulate a tip of a writing instrument, which can make use of the device 600 more intuitive for a user.

The distal tip 602 in this illustrated embodiment is configured to be removably and replaceably attachable to the body, e.g., to a distal end of the first body portion 604. The distal tip 602 and the first body portion 604 can be configured to be removably and replaceably attached to one another in a variety of ways. As in the illustrated embodiment, a proximal end of the distal tip 602 can be configured to releasably mate with a distal end of the first body portion 604. The first body portion's distal end can include an attachment mechanism configured to mate with a corresponding attachment mechanism of the distal tip 602. Examples of the attachment mechanisms include those discussed above with respect to the distal tip 402 releasably mating to the body of the device 400 of FIG. 5. In this illustrated embodiment, as shown in FIG. 16, the distal tip 602 and the first body portion 604 can be configured to be releasably matable via snap fit. The first body portion's attachment mechanism can include a depression 626 formed therein, and the distal tip's attachment mechanism includes a protrusion 628 configured to be seated in the depression 626. The protrusion 628 in this illustrated embodiment includes a raised circumferential ridge extending around an internal surface of a cavity 630 formed in the proximal end of the distal tip 602. The depression 626 in this illustrated embodiment includes a circumferential indentation extending around an external surface of a lip 632 extending from the distal end of the first body portion 604. The lip 632 can include the viewing element 618 at a distal end thereof. The lip 632 can include a reduced diameter region of the first body portion 604. The lip 632 can be configured to be disposed within the cavity 630 when the distal tip 602 and the first body portion 604 are releasably mated together, e.g., when the distal tip 602 is snap fit to the first body portion 604 with the protrusion 628 snapped into the depression 626. The viewing element 618 can thus be configured to be disposed at least partially within the distal tip 602 when the distal tip 602 and the first body portion 604 are attached together. The distal tip 602 can help protect the viewing element 618 from damage when the distal tip 602 and the first body portion 604 are attached together and/or can help position the viewing element 618 closer to a target when the distal tip 602 is tracing.

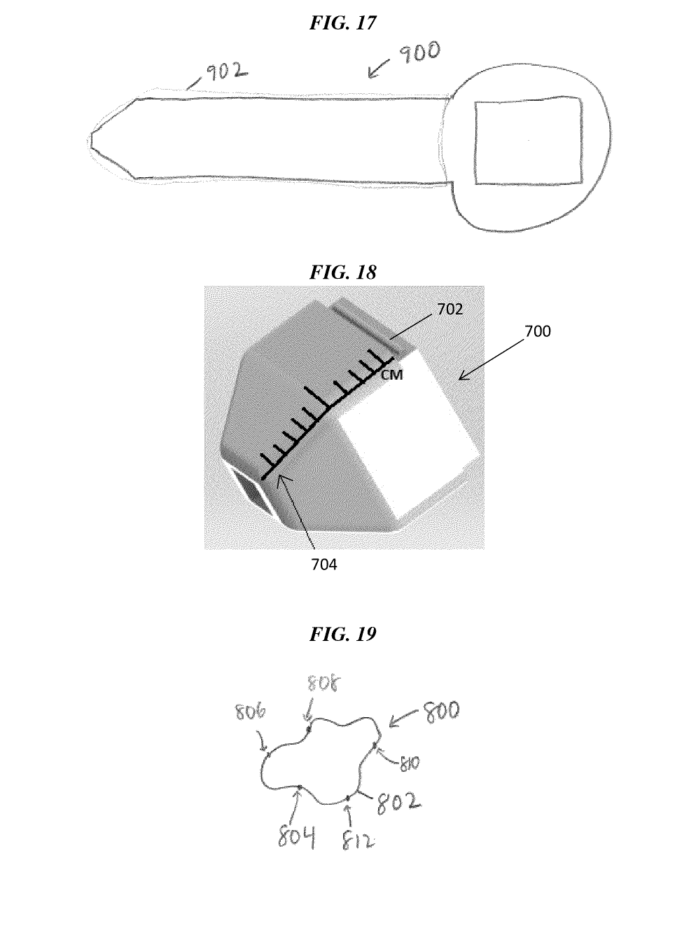

FIG. 17 illustrates another embodiment of a handheld device 900 configured to trace a perimeter of a wound of a patient so as to allow a size of the wound to be determined. The handheld device 900 can include a housing that includes a body and a non-removable distal tip. At least a distal portion of the device 900 can be configured to be encased in a protective cover 902. The protective cover 902 can be sterile and can be configured to provide a sterile tracing portion of the device 900. The protective cover 902 can include a sleeve, sheath, or bag, and can be configured and used similar to a thermometer sheath. The protective cover 902 can be substantially transparent in at least a portion thereof adjacent the distal tip so as to facilitate data gathering via the distal tip. The protective cover 902 can be removably and replaceably attachable to the device 900, e.g., by sliding on and off a shaft portion of the device, which can facilitate reuse of the device 900 and/or facilitate disposability of the protective cover 902.

A handheld device can be configured to provide a depth measurement. For example, in wound applications, the distal tip can be configured to be inserted into a wound and indicate a depth of the wound. The depth measurement can facilitate evaluation of the wound's severity and/or prognosis. A series of depth measurements of the wound taken at different times, e.g., on different days, can facilitate evaluation of the wound's healing progress and/or facilitate determination of appropriate treatment for the wound. The depth measurement can facilitate determination of 3D sizes, such as volume, by providing z dimension measurement data.

FIG. 18 illustrates an embodiment of a distal tip 700 of a handheld device configured to provide a depth measurement. The distal tip 700 in this illustrated embodiment is configured to be removably and replaceably attached to a body of a handheld device, but distal tips configured to provide a depth measurement can instead be permanently attached to a handheld device's body. Additionally, the distal tip 700 includes a protrusion 702 configured to facilitate snap fit of the distal tip 700 to a body of a handheld device, but distal tips configured to provide a depth measurement can be configured to be removably and replaceably attached to a body of a handheld device in a way other than snap fit using the protrusion 702.

The distal tip 700 in this illustrated embodiment includes indicia 704 formed thereon that is configured to facilitate depth measurement. The indicia 704 can be printed, embossed, etched, etc. on the distal tip 700. The indicia 704 can include a plurality of measurement lines, similar to a ruler. The indicia 704 in the illustrated embodiment is in centimeters, but indicia can be provided in any one or more units of measure. The distal tip 704 can be configured to be inserted into a wound or other item such that the indicia 704 can indicate a depth of the wound or other item by comparing a top surface of the wound or other item with the indicia 704. The distal tip 700 only includes one set of indicia 704, but a distal tip can include multiple sets of indicia, e.g., each set of indicia being in a different unit of measure, indicia being present on different sides of the distal tip, etc.

In some embodiments, a proximal tip of a handheld device can be configured to provide a depth measurement, similar to that discussed above regarding a distal tip being so configured. In this way, a distal tip of the device can be configured to trace a perimeter of a wound of a patient so as to allow a size of the wound to be determined, and the proximal tip of the device can be configured to allow a depth of the wound to be determined. The proximal tip can be integrally formed with the device, e.g., be permanently attached thereto, or the proximal tip can be removably and replaceably attached the device at a proximal end thereof. By being removably and replaceably attached the device, the proximal tip can be configured to be disposable, e.g., used once and/or only with one patient. A handheld device's proximal and distal tips can both be permanently attached to the device, the proximal and distal tips can both be removably and replaceably attached to the device, or one of the proximal and distal tips can be permanently attached to the device while the other of the proximal and distal tips can be permanently attached to the device.

In some embodiments, a handheld device can include an image gathering device, e.g., a still image camera, a video image camera, an ultrasound range finder, etc., configured to facilitate depth measurement. The image gathering device can be configured to gather one or more images of a target, e.g., of a wound, when the handheld device is positioned adjacent thereto. A processor, on-board and/or off-board, can be configured to analyze the gathered image(s) so as to determine a depth of the target.

A kit can include a handheld device body and a plurality of distal tips each configured to mate to the body, thereby providing for optimal selection of distal tips to mate to the body. Each of the distal tips can have a size and/or shape different from at least one other of the distal tips, which can facilitate selection of a specific distal tip appropriate for use in a particular situation, e.g., for a particular wound size, for a particular wound location, etc. In an exemplary embodiment, the kit can include only one handheld device body, which can help reduce a monetary cost of the kit since a handheld device body that includes electronics components can be more costly than a distal tip that lacks electronics components. A kit can, however, include a plurality of handheld device bodies, with or without also including a plurality of distal tips. In some embodiments, a kit can be provided that includes a plurality of distal tips each configured to mate to a handheld device body, but the kit can be provided without the body. In other words, a pack of replacement distal tips can be provided.

In use, as mentioned above, the handheld devices disclosed herein can be used to measure a size of a wound. For example, a handheld device can be used in a surgical procedure to measure a size of a wound in the form of an incision made in the patient's skin. The size of the incision can facilitate determination of, e.g., what size trocar and/or other surgical tool to insert through the incision. For another example, a handheld device can be used to measure a size of a wound in the form of a pressure ulcer on a skin surface of a patient. The size of the wound can facilitate determination of, e.g., how well the pressure ulcer is healing based on a comparison of sizes of the wound at different points in time.

FIG. 19 illustrates one example of a wound 800 whose size can be determined using a handheld device. Although the process of determining the size of the wound 800 is discussed below with respect to the handheld device of FIGS. 3 and 4, any of the handheld devices disclosed herein can be used to measure the size of the wound 800. Additionally, the wound 800 in the illustrated embodiment includes a pressure ulcer, but as mentioned above, any type of wound can be measured using a handheld device disclosed herein.

If the device is not powered on, the handheld device can be turned on by pressing the power button 308, e.g., by a user manually pressing the power button 308. The processor 330 can be configured to cause the display screen 316 to indicate that the device is “on” in any one or more ways, e.g., by lighting up the screen 316, by displaying the text “ON” on the screen 316, by showing a power icon on the screen 316, etc. The device can be immediately ready for use, or the device may require a brief warm-up time to reset and calibrate. The user can push the reset button 310 to ready the device for data gathering. In an exemplary embodiment, the reset button 310 need not be pressed before the device is first used to gather data after being turned on. The processor 330 can be configured to cause the display screen 316 to indicate when pushing of the reset button 310 is required, e.g., by displaying a warning message on the screen 316, by showing a reset icon on the screen 316, etc.

If a handheld device with a removable distal tip is being used, and the distal tip is not already attached to the device's body when the device is “on” and otherwise ready for data gathering, the distal tip can be attached to the body. Additionally, if a plurality of distal tips are available for attachment to the device's body, a one of the distal tips having an appropriate size and shape for the wound 800 can be selected from among the distal tips and attached to the body. If a distal tip is not attached to the body, the processor can be configured to cause a notification signal to be provided to the user indicating that a distal tip needs to be attached to the body. Examples of the notification signal include a message, icon, etc. on a display screen, a sound, etc.

If a handheld device with a removable power source is being used, and the power source is not already attached to the device's body when the device is ready to be turned on, the power source can be attached to the body. Similarly, if a handheld device without an on-board power source is being used, the handheld device can be coupled to a power source, e.g., plugged into a wall outlet.

To prepare the device for tracing the wound 800, the distal tip 302 can be positioned adjacent the wound 800 with the viewing window 318 being positioned above a perimeter 802 of the wound 800, e.g., above a surface of the skin in which the wound 800 is formed. The distal tip 302 can directly contact the perimeter 802, although direct contact is not needed when using optical data gathering as in the illustrated embodiment or when using other types of light-based data gathering such as laser data gathering. As mentioned above, by not directly contacting the perimeter 802 or any other portion of the wound 800, the handheld device will not irritate or otherwise damage the wound 800, will not cause the patient pain, will not open the wound 800 and cause bleeding, etc.

When the viewing window 318 is positioned above the wound's perimeter 802, the read button 312 can be pressed, e.g., manually pressed by the user. Pressing the read button 312 can indicate that the device is at a start position 804 and can trigger a start of data gathering. The start position 804 can be anywhere around the perimeter 802. The triggered start can cause the processor 330 to open the optical navigation chip 326 so as to ready the viewing element 320 to begin gathering optical data. The triggered start can also cause the processor 330 to gather first coordinate data using the viewing element 320. The first coordinate data can indicate a “home” or a reference point for subsequently gathered coordinate data during the tracing of the wound's perimeter 802. In other words, the first coordinate data can establish a relative coordinate system by defining a zero point to which subsequently gathered coordinate data can be referenced. In an exemplary embodiment, the coordinate data at the start position can be represented as (0,0) in (x,y) format. In some embodiments, the coordinate data can also include a third dimension of data, e.g., also include z dimension data, such that the start position can be represented as (0,0,0) in (x,y,z) format.

After having pressed the read button 312, and hence after the start position 804 has been identified via the first coordinate data, the device can be moved around the perimeter 802, thereby tracing the perimeter 802 with the distal tip 302. The perimeter 802 can be traced clockwise or counterclockwise. When the perimeter 802 is being traced, the processor 330 can be configured to cause the device to gather a series of coordinate data one after another, such as by gathering data at predetermined time intervals, e.g., every one second, every 10 μs, etc. The predetermined time interval can be so short that the processor 330 essentially causes continuous data gathering, with sets of coordinate data being gathered immediately after one another. The predetermined time interval can be preprogrammed into the memory. Because different users may move the device at different speeds, the predetermined time interval can be relatively short so as to help maximize a number of coordinate data that is gathered as the device moves around the perimeter 802, regardless of the user's speed. The processor 330 can cause any number of coordinate data to be gathered around the perimeter 802 of the wound 800, such as at each of a plurality of sequential positions 806, 808, 810, 812 around the perimeter 802. Only five positions 804, 806, 808, 810, 812 where coordinate data is gathered are shown in the illustrated embodiment, but in an exemplary embodiment, more coordinate data would be gathered around the perimeter 802 in order to provide a more accurate size determination. By way of example, the coordinate data for the second position 806 can have a negative x value and a positive y value with respect to the first, start position 804, the coordinate data for the third and fourth positions 808, 810 can each have positive x and y values with respect to the first, start position 804, and the coordinate data for the fifth position 812 can have a positive x value and a negative y value with respect to the first, start position 804.

The gathered data can be communicated, e.g., wirelessly transmitted, automatically to an external storage unit. In other words, the gathered data can be streamed while it is being gathered. Such streaming can be particularly useful if the device does not include an on-board memory.

The processor 330 can be configured to associate the gathered data with a specific patient, e.g., the patient that has the wound 800. For example, the device can be configured to scan a barcode on a patient's wristband or paper chart and store the scanned data as patient identification information in the device on-board memory and/or in an external memory. The processor 330 can be configured to tag the gathered data as being associated with that patient identification information. For another example, the device can be configured to accept a manual input from a user indicative of a specific patient, such as by the screen 316 being a touchscreen on which the user can enter the patient's name, identification number, etc. The processor 330 can be configured to tag the gathered data as being associated with that manually input patient identification information.

If a handheld device includes one or more angular measurement mechanisms, the device can be configured to provide an angular indicator signal to the user. Examples of the angular indicator signal include a sound, text on the display 316, and an image on the display 316. The angular indicator signal can provide notification to the user that the device is positioned relative to the target, e.g., the wound 800, at an inappropriate angle at which the viewing element 318 likely cannot properly visualize the perimeter 802. The device can be configured to provide the angular indicator signal, e.g., sound a tone, display a message on the screen 316, etc., until the device is positioned relative to the target at an angle at which the viewing element 318 likely can properly visualize the perimeter 802. Alternatively, the angular indicator signal can provide notification to the user that the device is positioned relative to the target, e.g., the wound 800, at an appropriate angle at which the viewing element 318 likely can properly visualize the perimeter 802, e.g., illuminate a light indicating that the device is properly positioned for tracing the target.

When the device has traced an entirety of the perimeter 802 so as to be back at the start position 804, the user can actuate the read button 312 a second time, thereby signaling that the entire perimeter 802 has been traced. The user can generally be relied upon to press the read button 312 when the viewing element 320 returns to the start position 804, but the read button 312 may not be pressed for the second time when the viewing element 320 is exactly at the start position 804 where the read button 312 was first pressed. The processor 330 can be configured to compare the last coordinate data gathered, or a plurality of gathered coordinate data at the very end of the gathered coordinate data series, with the coordinate data for the start position 804. If the last coordinate data gathered, or any of the plurality of gathered coordinate data at the very end of the gathered coordinate data series, includes the zero point, e.g., has coordinate data of (0,0), then the processor 330 can be configured to exclude coordinate data after that second gathered zero point from size determinations. In other words, the processor 330 can be configured to determine when an overlapping portion of the perimeter 802 has been traced because the user traced more than the complete perimeter 802.

In some embodiments, a handheld device may not include a read button. In such a case, a start of tracing can be triggered when a distal tip of the device directly contacts a target, e.g., when a distal tip directly contacts a skin surface. Similarly, a stop of the tracing can be triggered when the distal tip ceases to directly contact the skin surface. The direct contact can be determined in a variety of ways, as will be appreciated by a person skilled in the art, such as by using a pressure sensor.

The tracing can, but may not, precisely follow the perimeter 802 based on a particular user's precision in user of the device. For wound measurement, consistency between wound measurements performed at different times, e.g., on different days, can be more important than accuracy of a specific wound measurement. The same user can perform sequential wound measurements, e.g., measurements taken on consecutive days, or users trained to use the device in a same way, e.g., trained to trace just outside the actual perimeter 802, will all trace in substantially the same way. Thus, comparison between the wound sizes determined from each of the sequential wound measurements can accurately reflect any trends in the wound's size despite the specific wound measurements possibly not being actually reflective of the wound's actual size and/or being performed by different users. For example, if a wound is always traced just outside the wound's actual perimeter 802, as opposed to directly along the perimeter 802, sequential wound measurements will be comparing similarly gathered size data.

If at any point during the tracing around the perimeter 802 the user desires to start over, the user can reposition the device 802 at the start position 804 or at a new start position along the perimeter 802 and can push the reset button 310. The user can then push the read button 312 to begin the tracing process anew, with the processor 330 triggering a new start of data gathering.

The processor 330 can be configured to determine a size of the wound 800 using the gathered coordinate data. In an exemplary embodiment, the processor 330 can be configured to calculate, using traditional mathematical formulas, a total surface area of the wound 800 and a total perimeter length of the wound 800 using the gathered coordinate data. The processor 330 can cause the calculated total surface area and the calculated total perimeter length to be displayed on the screen 316, to be stored on-board, and/or communicated, e.g., wirelessly communicated, to an external device for processing and/or storage. The data can be communicated on demand or automatically. The calculated size measurements can be displayed on the screen 316 in any one or more units of measure. The processor 330 can be configured to automatically display the calculated size measurements on the screen 316, or the processor 330 can be configured to display a user prompt asking whether the calculated size measurements should be displayed on the screen 316. The user can reply to the prompt by, e.g., touching the screen 316 if the screen 316 is a touchscreen.

If the handheld device includes one or more angular measurement mechanisms, the processor 330 can be configured to account for angular orientation of the device at each of the positions 804, 806, 808, 810, 812. In this way, the processor 330 can be configured to account for angular adjustments of the device made during the tracing, which could result in variances of coordinates relative to the zero point that do not reflect accurately actual coordinates of the perimeter 802.

The processor 330 can be configured to cause the display screen 316 and/or an external display in communication with the device to show a simulation of the wound based on the determined size. The simulation can be helpful, for example, in explaining wound information to a patient, a patient's family, etc., and/or in detecting abnormalities in size progression between subsequently taken wound measurements. For example, the processor 330 can be configured to draw a graphical outline of the wound 800 on the screen 316 based on the gathered coordinate data. If the device also gathered image data, the graphical outline can be overlaid on a gathered image of the wound 800, thereby allowing accuracy of the tracing to be visually evaluated by a user.

If the handheld device has image gathering capability, e.g., includes an HD camera, a video camera, etc., the processor 330 can be configured to cause one or more gathered images to be shown on the display screen 316 and/or on an external display in communication with the device. The gathered image(s) can be displayed on demand, or can be automatically displayed, such as in a gallery format. A plurality of gathered still images from a series of wound measurements taken at different periods of time, e.g., on different days, can be displayed in a time lapse format so as to provide a visual indication of a wound's healing progress. The gathered images can be associated with coordinate data gathered at a same time, which can facilitate proper orientation of the images.

If the handheld device is configured to gather data regarding one or more parameters in addition to or instead of size, e.g., temperature, pH, chemical or biological composition, moisture content, etc., the processor 330 can be configured to store, analyze, and/or display the other parameter data similar to that discussed herein regarding gathered size data. The gathered parameter data can be associated with coordinate data gathered at a same time, which can facilitate association of the parameter data with a specific part of the wound 800. The processor 330 and/or an external processor can be configured to analyze gathered non-size data so as to determine one or more useful therapeutic and/or diagnostic factors. For example, the processor 330 and/or the external processor can be configured to analyze gathered chemical or biological composition data regarding the wound so as determine which of a plurality of predetermined wound types, each having an associated chemical or biological composition, the wound most closely matches. For another example, processor 330 and/or the external processor can be configured to analyze healing progression/regression of a wound by comparing gathered non-size data with previously gathered non-size data for the wound, e.g., by comparing current enzyme/protein content of the wound with previous enzyme/protein content of the wound.

When the perimeter 802 tracing has been completed and/or any desired calculated size data has been displayed and/or communicated externally, the power button 808 can be actuated to turn the device off. The processor 330 can be configured to automatically cause the device to turn off if the device is idle for a predetermined amount of time, e.g., fifteen minutes, five minutes, ten minutes, etc., as a power-saving feature. The device being idle can be based on, e.g., whether the read button 312 has been pushed within the predetermined amount of time.

The handheld devices disclosed herein can, as discussed above, be particularly useful in wound measurement applications. However, the handheld devices disclosed herein can have medical applications other than determining wound size and can have applications outside a medical context. The handheld device can be used similar to that discussed above regarding wound measurement, with size data (e.g., coordinate data) being similarly gathered and analyzed and/or with non-size data (e.g., color, moisture content, etc.) being similarly gathered and analyzed. If the handheld device is being used in a non-medical application, the handheld device (e.g., a distal tip thereof) need not be sterile.

Examples of non-wound measurement medical applications include classifying wounds (e.g., by gathering wound characteristic data such as color, depth, and chemical or biological composition with a handheld device for comparison with predetermined wound characteristics associated with different types and/or different wounds), measuring suture length (e.g., by tracing a handheld device along a longitudinal length of a suture), facilitating plastic surgery evaluations (e.g., before and after effects regarding skin elasticity and distance between facial features by tracing a handheld device along a skin surface between facial features), evaluating weight loss (e.g., measuring waist size by tracing a handheld device around a waist of a person and comparing measurements taken on different days), and evaluating skin grafts (e.g., determining color/tint matches by tracing a handheld device along a skin graft of a person and along non-grafted skin of the person and comparing the data gathered along the skin graft with the data gathered along the non-grafted skin).

Examples of applications outside a medical context include taking measurements in architectural drawings (e.g., by tracing design outlines with a handheld device), taking measurements of 3D models (e.g., by tracing a handheld device along a surface of the model), determining distances on a map (e.g., by tracing a route between start and end points with a handheld device), determining distances on a globe (e.g., by tracing a route between start and end points with a handheld device), determining sizes of construction components used by contractors and/or builders (e.g., by moving a handheld device along a length of a construction component to determine its length), determining effects of weightlifting and/or other exercise (e.g., by tracing a handheld device around a bicep and/or other body area to determine size and comparing measurements taken on different days), determining size and measurements of the human body for clothing purposes such as tailoring and custom-fit clothing (e.g., by tracing a handheld device along one or more areas of the body and using the obtained measurements to make clothing alterations and/or custom clothing designs), and educational assistance (e.g., helping students learn and/or perform geometric calculations such as perimeter and area by tracing a handheld device around a perimeter of a geometric shape on paper or on a display screen for comparison with a student's manual calculation).

A person skilled in the art will appreciate that the present invention has application in non-surgical medical procedures and in surgical medical procedures including conventional minimally-invasive and open surgical instrumentation as well application in robotic-assisted surgery.

The devices disclosed herein can also be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A device, comprising:
a housing configured to be handheld, a distal end of the housing being configured to trace a perimeter of a target tissue of a patient;
a sensor disposed within the housing and configured to collect coordinates at each of a plurality of discrete points around the perimeter when the distal end traces the perimeter, each of the coordinates being based on a position of the distal end relative to the target tissue at the discrete point at which the coordinates are collected; and
an output device disposed within the housing and configured to output at least one of the collected coordinates and a size of the target tissue that is based on the collected coordinates.

2. The device of claim 1, wherein each of the coordinates includes (x,y) dimensional data, and the device further comprises:
a processor configured to calculate the size of the wound using the (x,y) dimensional data for each of the coordinates, the size of the target tissue including at least one of a surface area enclosed by the perimeter and a length of the perimeter.

3. The device of claim 2, wherein each of the coordinates also includes (z) dimensional data, and the processor is configured to calculate the length of the perimeter using the (x,y,z) dimensional data for each of the coordinates.

4. The device of claim 2, wherein:
the distal end has indicia formed thereon that is configured to provide (z) dimensional data,
the target tissue includes a wound,
the indicia is configured to provide the (z) dimensional data when the distal end is inserted into the wound, and
the processor is configured to calculate the size of the wound using the (z) dimensional data and using the (x,y) dimensional data for each of the coordinates, the size of the wound including at least a volume of the wound.

5. The device of claim 1, wherein the distal end has indicia formed thereon that is configured to indicate a depth of the target tissue when the distal end is inserted into the target tissue.

6. The device of claim 1, wherein the output device includes a communication mechanism that is disposed within the housing, the communication mechanism being configured to wirelessly communicate the at least one of the collected coordinates and the size of the target tissue to a computer that is separate and remote from the device.

7. The device of claim 1, wherein the output device includes at least one of a display screen and an audio speaker,
the display screen being visible on a surface of the housing, and the display screen being configured to display the at least one of the collected coordinates and the size of the target tissue, and
the audio speaker being configured to audibly provide the at least one of the collected coordinates and the size of the target tissue.

8. A system, comprising:
the device of claim 1; and
a processor configured to calculate the size of the target tissue based on the collected coordinates.

9. A kit, comprising:
the device of claim 1; and
a plurality of distal tips configured to be removably and replaceably attached to the housing, the housing being configured to have one of the distal tips attached thereto at a time, and the distal end of the housing being one of the distal tips.

10. A device, comprising:
a housing configured to be handheld, a distal end of the housing being configured to move along a surface of a target so as to make an invisible line along the surface;
a sensor disposed within the housing and configured to collect coordinates at each of a plurality of discrete points along the invisible line when the distal end moves along the surface, each of the coordinates being based on a position of the distal end relative to the target at the discrete point at which the coordinates are collected; and
an output device disposed within the housing and configured to output at least one of the collected coordinates and a size characteristic of the target that is calculated from the collected coordinates.

11. A method, comprising:
gathering coordinate data by making an invisible perimeter line around a complete perimeter of a target tissue of a patient with a sterile distal end of a handheld device so as to collect a series of coordinates around the complete perimeter of the target tissue;
calculating a size of the target tissue using the gathered coordinate data; and
storing the size in an electronic memory.

12. The method of claim 11, wherein each of the coordinates in the series of coordinates includes (x,y) dimensional data of the target tissue, the calculating using the (x,y) dimensional data for each of the coordinates, and the size includes at least one of a surface area of the target tissue and a length of the complete perimeter.

13. The method of claim 11, wherein each of the coordinates in the series of coordinates includes (x,y,z) dimensional data, the calculating using the (x,y,z) dimensional data for each of the coordinates, and the size including the length of the complete perimeter.

14. The method of claim 11, further comprising measuring a depth of the target tissue, wherein:
the calculating also uses the measured depth; and
the size includes a volume of the target tissue.

15. The method of claim 11, further comprising removing the sterile distal end from a body of the handheld device;
attaching a second sterile distal end to the body;
gathering additional coordinate data by making a second invisible perimeter line around the complete perimeter of the target tissue with the second sterile distal end attached to the body;
calculating a second size of the target tissue using the gathered additional coordinate data; and
storing the second size in the electronic memory.

16. The method of claim 11, further comprising removing a portion of the handheld device that includes the electronic memory from a body of the handheld device; and
after the removal, re-attaching the portion of the handheld device to the body of the handheld device.

17. The method of claim 16, further comprising, after the removal and before the re-attaching, attaching the portion of the handheld device to a computer, thereby allowing the computer to access to stored wound size.

18. The method of claim 11, further comprising wirelessly communicating the stored size from the electronic memory to a second electronic memory that is separate and remote from the handheld device.

19. The method of claim 11, wherein storing the size comprises wirelessly communicating the gathered size data to a computer that is separate and remote from the handheld device.

20. The method of claim 11, further comprising, after a passage of time from gathering the coordinate data, gathering second coordinate data by making a second invisible perimeter line around the complete perimeter of the target tissue with at least one of the handheld device and a second sterile distal end of a second handheld device;
calculating a second size of the target tissue using the gathered second coordinate data; and
determining a change in a size of the target tissue between a time when the coordinate data was gathered and a time when the second coordinate data was gathered by comparing the size and the second size.
